(19) United States
(12) Patent Application Publication
(10) Pub. No.: US 2019/0015445 A1
(43) Pub. Date: Jan. 17, 2019

(54) INJECTION CONTAINING CHLORINE DIOXIDE AND METHOD FOR MAKING SAME

(71) Applicant: Xuewu LIU, (US)

(72) Inventors: Xuewu LIU, Shenzhen (CN); Xueyan LIU, Shenzhen (CN)

(21) Appl. No.: 16/055,039

(22) Filed: Aug. 4, 2018

Related U.S. Application Data
(63) Continuation of application No. PCT/CN2017/071589, filed on Jan. 18, 2017.

(30) Foreign Application Priority Data
Mar. 8, 2016 (CN) 201610126960.1

(51) Int. Cl. A61K 33/00; A61K 9/00; A61P 35/00

(52) U.S. Cl. CPC A61K 33/00; A61P 35/00; A61K 9/0019

(57) ABSTRACT

Disclosed is an injection containing chlorine dioxide in therapeutic applications such as in-vivo stem cell regeneration, anti-tumor and anti-aging. The chlorine dioxide injection of the present invention has a high pharmacological effect and a low toxic or side effect, enabling ablation of tumors and promotion of in-situ tissue regeneration. Particularly, the chlorine dioxide injection stimulates an immune response through the ablation of target tumors, causing the immune system to inhibit or eliminate distal tumors or metastatic tumors. A method for making the chlorine dioxide injection is further provided.

### CROSS-REFERENCE TO RELATED APPLICATIONS

This application is a continuation of International Application No. PCT/CN2017/071589 with a filing date of Jan. 18, 2017, designating the United States, now pending, and further claims priority to Chinese application no. 201610126960.1 with a filing date of Mar. 08, 2016. The content of the aforementioned applications, including any intervening amendments thereto, are incorporated herein by reference.

### TECHNICAL FIELD

The present invention relates to an injection containing chlorine dioxide in therapeutic applications such as in-vivo stem cell regeneration, anti-tumor and anti-aging, and in particular to an injection preparation with a high pharmacological effect and a low toxic or side effect and capable of long-term storage, and a method for making the same.

### BACKGROUND OF THE PRESENT INVENTION

Chinese Patent Application Publication No. CN103040860A discloses that direct administration of an injection containing chlorine dioxide may initiate regeneration of stem cells in-vivo to treat the related diseases. Chinese Patent Application Publication No. CN103720709A discloses that it is possible to clear senescent cells of target tissues and ablated tumor cells with direct administration. As disclosed in CN103720709A, an injection containing chlorine dioxide is administrated to function as anti-aging and anti-tumor preparation that is, for the treatment of aging and cancers. Both of the above documents disclose the use of a chlorine dioxide solution as an injection for the treatment of the related diseases. However, generally, if the injection is to be stored as a solution for a long time, the concentration of gaseous chlorine dioxide dissolved in the solution is limited; if the injection is to be prepared on site before use, the chlorine dioxide precursor and other reactants cannot be removed, so that more impurities will be included in the prepared chlorine dioxide solution, and as a result, the solution in the form of an injection may not meet the safety requirements.

Generally, the dissolution of chlorine dioxide in an aqueous solution is very limited. The solubility of chlorine dioxide at 20° C. is about 2.9 g/L. If chlorine dioxide is dissolved in a saline solution, the solubility of chlorine dioxide is lower than that of chlorine dioxide in pure water. Therefore, in the prior art, it is difficult to prepare a chlorine dioxide solution having a concentration of more than 3 mg/ml and store the chlorine dioxide solution for a long period of time. However, when the chlorine dioxide solution is used as a disinfectant for disinfection, a high concentration of chlorine dioxide solution is generally not required, so it is unnecessary to prepare a high concentration of chlorine dioxide solution in the past. When the chlorine dioxide solution is used as an injection, those skilled in the art have never thought of increasing the concentration of the chlorine dioxide solution. For example, the preparations containing chlorine dioxide, as disclosed in the above two patent applications, are prepared in a method in which the chlorine dioxide gas is produced by preparing chlorine dioxide gas having a concentration of 99.9% or more (preferably, by reacting chlorite with an acid). The chlorine dioxide gas is bubbled and dissolved in an aqueous solution to obtain 500 ppm to 5900 ppm (0.05% w/w, 2.9 mg/ml) of chlorine dioxide solution. The solution can be used as an injection containing only chlorine dioxide and additives commonly used in injections.

In addition, the chlorine dioxide solution is typically prepared on site. Chinese Patent No. CN103402621A discloses a method for preparing a high concentration of stable chlorine dioxide solution by mixing dithionate with chlorite. More generally, a chlorine dioxide solution is made on site by mixing a chlorine dioxide precursor (e.g., sodium chlorite) with an acid to generate chlorine dioxide, wherein the chlorine dioxide is directly dissolved in the mixed solution and can be maintained at a high concentration, e.g., 2% (w/w) in a short period. The chlorine dioxide may be used by dilution or used without dilution. In this case, in addition to the chlorine dioxide, the mixed solution further contains other impurities. This will not bring about adverse effects in the general use of such solution; however, as an injection this may introduce risk factors. Usually, an excessive amount of acid is added to allow for a complete reaction, resulting a lower pH of the mixed solution which may not be suitable for use as an injection to be injected into the human body.

### SUMMARY OF THE PRESENT INVENTION

It has been found by experiments that an injection containing a low concentration of chlorine dioxide cannot provide remarkable therapeutic effects, for example in promotion of regeneration, anti-aging, cancer treatment, or shows no significant therapeutic effects compared to an injection containing a high concentration of chlorine dioxide (e.g., 0.5-20 mg/ml of chlorine dioxide). In addition, production of a chlorine dioxide solution having a concentration of about 2% (w/w) in conventional means will bring the unreacted chlorine dioxide precursor and other reactants to the chlorine dioxide solution. For example, the chlorine dioxide solution which is prepared by mixing sodium chlorite with citric acid contains sodium chlorite, sodium citrate, sodium chloride etc., in addition to chlorine dioxide. This may not be an isotonic solution, and excessive impurities included herein will make the injection unsafe. Meanwhile, pH of the solution may be too low due to excessive acid which will bring a safety risk.

The following defects of the prior art have been found: chlorine dioxide injections with higher safety are less obvious or low in pharmacological effect due to a lower concentration of chlorine dioxide; while chlorine dioxide injections with higher pharmacological effect have obvious toxic or side effects due to various impurities and low pH caused by the mixed reaction for generating chlorine dioxide. The present invention provides a solution to address the above problems.

In view of the defects of the prior art, there is a need to develop an injection containing chlorine dioxide, which reaches a compromise between pharmacological effect and toxic or side effect of the chlorine dioxide preparation, thereby improving the pharmacological effect of the chlorine dioxide preparation and reducing its toxic or side effect. More particularly, such injection comprises a high concentration of chlorine dioxide without any additional impurities, and the pH is also acceptable for the injection. Apparently, a common method is to dissolve more chlorine dioxide in an injection solution. However, it is well-known that the solubility of chlorine dioxide at room temperature is only 2.9 g/L, and thus the production of an injection containing a higher concentration of chlorine dioxide requires more creative work.

In order to realize more remarkable pharmacological effect of chlorine dioxide and eliminate the disadvantages of impurities and low pH, intensive studies and experiments have been conducted. The results show that i) chlorine dioxide gas can be prepared by a method for preparing chlorine dioxide gas having a concentration of 99.9% or more; ii) by introducing the chlorine dioxide gas into a low-temperature medium (for example, into air below 11° C., or bubbling the chlorine dioxide gas into a cold saline solution below 11° C. or a solution containing other pharmaceutically acceptable additives commonly used in injections) and then dissolving the chlorine dioxide in the low-temperature medium, the concentration of the dissolved chlorine dioxide is not limited by the solubility of the chlorine dioxide. In this way, an injection containing a high concentration of chlorine dioxide is prepared without irrelevant impurities and having a neutral pH.

In addition, it has been surprisingly found that the injection containing a high concentration of chlorine dioxide of the present invention not only produce a pharmacological effect in the vicinity of the injected tissues, but also stimulate immune response of the immune system, for example by ablation of tumors, to inhibit or eliminate distal tumors or metastatic tumors. This pharmacological phenomenon is unexpected for those skilled in the art, that is, the injection plays a positive role in the immune system and exerts a systemic pharmacodynamic effect through the immune system.

The present invention provides a chlorine dioxide solution (injection) having a concentration of chlorine dioxide exceeding its solubility; wherein the concentration of chlorine dioxide in the injection is 3 mg/ml, i.e., 100% (w/w), and is free of other substances (i.e., impurities) caused during the reaction for generating chlorine dioxide gas. Here, the impurities include chlorine dioxide precursors, acids and other reactants. For example, when sodium chlorite and citric acid are reacted to generate chlorine dioxide gas, the chlorine dioxide injection of the present invention does not contain additional impurities which are directly caused by the reaction, such as sodium chlorite, citric acid and sodium citrate. In the chlorine dioxide injection, the concentration of chlorine dioxide is preferably 5-25 mg/ml, more preferably 8-20 mg/ml. The injection contains only chlorine dioxide and additives which are commonly used in injections.

The chlorine dioxide solution is stored as an injection away from light at −20° C. to 11° C. for a long period of time. Preferably, the chlorine dioxide solution is stored away from light at −18° C. to 9° C. More preferably, the chlorine dioxide solution is stored away from light at 2° C. to 8° C.

The chlorine dioxide solution as an injection may be diluted to a target concentration before use with a dilute solution containing pharmaceutically acceptable additives commonly used in injections, and then injected into the body.

Further, the present invention provides a method for producing a chlorine dioxide injection with a high concentration of chlorine dioxide.

The method includes:
1) producing a chlorine dioxide gas having a concentration of 99.9% or more by a standard method;
2) introducing the chlorine dioxide gas into a medium at a low temperature of −20° C. to 11° C. via a duct; and
3) when the medium is a gaseous medium, collecting chlorine dioxide to obtain a chlorine dioxide injection with a concentration of chlorine dioxide approximate to 100%; and, when the medium is a liquid medium, raising temperature of the liquid medium containing chlorine dioxide to less than 20° C. to obtain the injection containing chlorine dioxide after the chlorine dioxide is uniformly dissolved in the liquid medium.

Methods for producing high-purity chlorine dioxide gas have been known by those skilled in the art; for example, reacting chlorine dioxide precursor (chlorite) and an acid to generate chlorine dioxide gas; or mixing and reacting dithionate with chlorite to generate chlorine dioxide gas.

In step 2, the chlorine dioxide gas may be directly introduced into a gas medium such as air or nitrogen, or can be bubbled into a dilute solution (e.g., saline solution) containing pharmaceutically acceptable additives that are commonly used in injections. Further, the chlorine dioxide is introduced into the low-temperature medium in a top-down approach. Particularly, at −20° C. to 0° C., a gas medium may be used; and at −2° C. to 11° C., a liquid solution that can be used as an injection may be used; for example, at 1° C. to 5° C., a saline solution is used.

The present invention further provides an injection containing a high concentration of chlorine dioxide and a method for preparing the injection. Conventionally, chlorine dioxide of a high concentration is a chlorine dioxide solution prepared by simply mixing various reactants, that is, various substances (especially chlorine dioxide precursors and acids) caused during the reaction for generating chlorine dioxide are not removed, or the pH of the solution is extremely low. As a result, the chlorine dioxide solution is unsafe for the human body when used as an injection. It has been proved that the generally prepared chlorine dioxide solution as an injection is highly toxic for the tested animals. Meanwhile, it has been found that the concentration of the chlorine dioxide solution prepared by introducing the generated chlorine dioxide gas into a solution is limited. At room temperature, the concentration is below 2.9 mg/ml. If this solution is used as an injection, the solution basically has no obvious pharmacological effect. The injection containing a high concentration of chlorine dioxide and the preparation method thereof provided by the present invention can effectively solve the two defects in the prior art, by which the pharmacological effect of chlorine dioxide is maximized without toxic or side effect due to the impurities and the low pH. In addition, it has been found that the injection containing a high concentration of chlorine dioxide of the present invention not only produce a pharmacological effect in the vicinity of the injected tissues, for example, ablation of tumors and promotion of in-situ tissue regeneration, but also stimulate the immune system, for example, ablation of tumors, stimulation of immune response, causing the immune system to inhibit or eliminate distal tumors or metastatic tumors.

### DETAILED DESCRIPTION OF THE PRESENT INVENTION

The present invention will be further described below in conjunction with embodiments.

The problem in the prior art has been found by experiments: existing injections containing chlorine dioxide contain a low concentration of chlorine dioxide so that the expected pharmacological effect cannot be achieved, or contain substances caused before or after the reaction for generating chlorine dioxide so that the injections for use in the human body are not safe. There are two methods for preparing an injection containing chlorine dioxide mentioned in the prior art.
1. The first method is to introduce the prepared high-purity chlorine dioxide gas into an aqueous solution. However, due to the limitation of the solubility, the prepared chlorine dioxide solution generally has a concentration of below 2.9 g/L. In one embodiment, it has been found by experiments that, in terms of pharmacological effect, an injection containing 2 g/L of chlorine dioxide is obviously less effective than an injection containing 15 g/L (or 15 mg/ml) of chlorine dioxide.
2. The second method is to mix a chlorine dioxide precursor (e.g., sodium chlorite) with an acid (e.g., citric acid) to produce a chlorine dioxide solution having a concentration of 1% to 10% (w/w) in a short period of time. In one embodiment, such chlorine dioxide injection having a concentration of 1.5% (w/w) has obvious toxic effect on the tested animals. In another embodiment, after the pH of the mixed solution is regulated to 7 by adding a pH regulator, the concentration of chlorine dioxide is reduced to 1% (w/w), but this injection still has obvious toxic effect on the tested animals.

The present invention provides an injection containing a chlorine dioxide solution having a concentration exceeding its solubility, wherein the concentration of the chlorine dioxide solution is 3 mg/ml, i.e., 100% (w/w). In the injection, the concentration of chlorine dioxide is preferably 5-25 mg/ml, more preferably 8-20 mg/ml. The injection contains only chlorine dioxide and additives which are commonly used in injections.

Further, the injection does not contain various chlorine dioxide precursors which are generally used for preparing chlorine dioxide or non-chlorine dioxide reactants. The chlorine dioxide precursors may include, for example, alkali metal chlorides and alkaline earth metal chlorides. The alkali metal chlorides may include, for example, sodium chlorite, potassium chlorite and lithium chlorite. The alkaline earth metal chlorides may include, for example, calcium chlorite, magnesium chlorite and barium chlorite. The non-chlorine dioxide reactants may include, for example, sodium citrate having a concentration of more than 1% of sodium chloride, potassium citrate or sodium sulfate.

In one embodiment, the injection containing a high concentration of chlorine dioxide in the present invention can directly ablate tumors, and can inhibit distal tumors and metastatic tumors by stimulating an immune response of the immune system. This indicates that the injection containing a high concentration of chlorine dioxide in the present invention not only has in-situ pharmacological effect on a target tissue, but also has pharmacological effect of building or promoting the immunity of the systemic immune system.

Further, the additives commonly used in injections are pharmaceutically acceptable solid or liquid carriers, for example, including but not limited to solvents, stabilizers, cosolvents, emulsifiers, clouding agents, buffer agents, isotonic agents, colorants, matrices, thickening agents, excipients, lubricants, adhesives, disintegrants, conditioners, foaming agents, super absorbent resin, surfactants, penetration enhancers, pH regulators, and the like.

Specifically, examples include: deionized water; sugars or sugar alcohols such as lactose, white sugar, fructose, glucose and mannose; cellulose such as crystalline cellulose, methylcellulose, ethyecellulose, hydroxypropyl cellulose, low-substituted hydroxypropyl cellulose, hydroxypropyl methyl cellulose, hydroxypropyl methyl cellulose phthalate, hydroxypropyl methylcellulose acetate succinate, hydroxymethyl cellulose, calcium carboxymethyl cellulose, sodium carboxymethyl cellulose, cross-linked sodium carboxymethyl cellulose, hydroxymethyl ethyl cellulose and cellulose acetate phthalate and derivatives thereof; starch such as corn starch, wheat starch, rice starch, potato starch, cyclodextrin and amylopectin, and derivatives thereof; natural polymers (algae, vegetable uncilage, proteins and the like) such as agar, sodium alginate, Arabic gum, gelatin, collagen, shell-lac, tragacanth gum and xanthan; synthetic polymers, such as polyvinylpyrrolidone, aminoalkylmethyl acrylic copolymer, methacrylic acid copolymer, hydroxyl vinyl copolymer, polyvinyl alcohol and dimethyl polysiloxane; oil such as olive oil, cocoa oil, carnauba wax, beef tallow, hardened oil, soybean oil, sesame oil, camellia oil, linseed oil, paraffin, liquid paraffin, beeswax, white vaseline, coconut oil and microcrystalline wax; fatty acids such as stearic acid, aluminum stearate, calcium stearate, magnesium stearate, triethyl citrate, glycerol triacetate, medium-chain fatty acid triglyceride, stearin and isopropyl myristate and derivatives thereof; alcohols and polyols such as ethanol, glycerol, stearyl alcohol, cetanol, propanediol and polyethylene glycol; inorganic substances and metal salt compounds such as zinc oxide, calcium hydrogen phosphate, precipitated calcium carbonate, synthetic aluminum silicate, silicic anhydride, kaoline, dried aluminum hydroxide gel, synthetic hrdrotalcite, titanium oxide, talc, bentonite, magnesium aluminum silicate, aluminum potassium sulfate, bismuth subgallate, bismuth subsalicylate, calcium lactate, sodium citrate, sodium chloride and sodium hydrogen carbonate; surfactants such as sucrose fatty acid ester, polyoxyl stearate, hydrogenated polyoxyethylene castor oil, polyoxypropylene polyoxypropylene glycol, sorbitan sesquioleate, sorbitan trioleate, sorbitan monstearate, sorbitan monopalmitate, sorbitan monolaurate, polysorbate, glycerin monostearate, sodium dodecyl sulfate and polidocanol; penetration enhancers such as dimethyl sulfoxide and analogues thereof, atone compounds, pyrrolidone derivatives, alcohol compounds and fatty acid compounds; pigments; perfumes; etc., but not limited thereto.

Further, the pH value of the injection is 4 to 8; preferably, 5 to 7.5.

Further, the chlorine dioxide solution is stored as an injection away from light at −20° C. to 11° C. for a long period of time. Preferably, the chlorine dioxide solution is stored away from light at −18° C. to 9° C. More preferably, the chlorine dioxide solution is stored away from light at 2° C. to 8° C.

A method for producing the injection containing a chlorine dioxide solution having a concentration exceeding its solubility of the present invention includes the following steps:
1) producing a chlorine dioxide gas having a concentration of 99.9% or more by a standard method; and
2) introducing the chlorine dioxide gas into a medium at a low temperature of −20° C. to 11° C. via a duct.

Methods for producing high-purity chlorine dioxide gas have been known to those skilled in the art; for example, reacting chlorine dioxide precursor (chlorite) and an acid to generate chlorine dioxide gas; or mixing and reacting dithionate with chlorite to generate chlorine dioxide gas.

In step 2, the chlorine dioxide gas may be directly introduced into a gas medium such as air or nitrogen, or can be bubbled into a dilute solution (e.g., saline solution) containing pharmaceutically acceptable additives that are commonly used in injections. Further, the chlorine dioxide is introduced into the low-temperature medium in a top-down approach. Particularly, at −20° C. to 0° C., a gas medium may be used; and at −2° C. to 11° C., a liquid solution that can be used as an injection may be used; for example, at 1° C. to 5° C., a saline solution is used

By controlling the time, a chlorine dioxide solution having a concentration of 3 mg/ml (i.e., 100% (w/w)) or pure chlorine dioxide can be prepared by the above steps. According to the actual requirements, the chlorine dioxide solution or pure chlorine dioxide may be diluted to a target concentration and then used as an injection for treating diseases.

The injection containing chlorine dioxide of the present invention may be administrated by any means of administration to a desired tissue. For example, the administration of the injection may be intravenous, intravenous, intraarterial, intramuscular, subcutaneous, intradermal, intracardiac, intraperitoneal, intrathecal, intra-articular, puncture, rectal, sublingual, nasal, transdermal, via inhalation or topical to a target organ or tissue, etc., but is not limited thereto.

Any of the preparations in General Rules on Preparations in China Pharmacopoeia can be used as an extended dosage form of the injection containing chlorine dioxide in the present invention. In the method of the present invention, examples of the dosage form of the injection containing chlorine dioxide as a drug include: injections (including suspensions and emulsions) directly applied to the body; ointments (including grease ointment, emulsion ointment (cream) or water-soluble ointment); inhalant, liquid preparations (including eye drops or nasal drops), suppositories, patches, cataplasms or lotions for external use; or tablet s(including sugar coatings, films and gel coatings), capsules, granules, powders (including fine-graded powders), pills, syrups and lozenges; and the like.

### EMBODIMENTS

### Embodiment 1: Preparation of an Injection Containing 15 mg/ml of Chlorine Dioxide

28% of sodium chlorite solution and 50% of citric acid solution were mixed in a reactor, and then introduced to a saline solution containing 0.9% of sodium chloride at 1° C. via a duct, with an inlet of the duct immersed in the saline solution. The reactor was heated at a constant temperature of 85° C. for 30 minutes, and chlorine dioxide was bubbled into the saline solution which was always maintained at the constant temperature of 1° C. by an external device. After 30 minutes, the mixed solution was transferred to a closed container and then placed at less than 20° C. After the chlorine dioxide and the saline solution were mixed uniformly, the mixed solution was placed in a refrigerator at 2° C. to 8° C. for storage. By measurements, the chlorine dioxide solution has a concentration of 15 mg/ml. The solution may be packaged in 10 ml glass bottles as injections, and then stored away from light at 2° C. to 8° C.

### Embodiment 2: Toxicity Tests of the Injections Containing Chlorine Dioxide

Preparation of the injection containing chlorine dioxide: injection A having a concentration of about 1.5% was prepared by mixing a chlorine dioxide precursor with an acid. Specifically, a mixed solution containing sodium chlorite of a concentration of 7.47% and sodium chloride of a concentration of 1.59% was prepared with deionized water to obtain a first solution; and a citric acid solution having a concentration of 16.7% was prepared with deionized water to obtain a second solution. The same volume of solutions were taken from the containers of different solutions, mixed, and the solution was allowed to stand still for 3 to 5 minutes. By measurements, the concentration of chlorine dioxide was about 1.5%, the pH was about 2.1, and was to be used for injection within 30 minutes. Injection B is obtained by mixing a chlorine dioxide precursor with an acid and then adding sodium hydroxide to regulate the pH to 7. A mixed solution containing sodium chlorite having a concentration of 7.47% and sodium chloride having a concentration of 1.59% was prepared with deionized water to obtain a first solution; and a citric acid solution having a concentration of 16.7% was prepared with deionized water to obtain a second solution. The same volume of solutions were taken from the containers of different solutions, mixed, and the solutions were allowed to stand still for 3 to 5 minutes. 10% of sodium hydroxide solution was added to adjust the pH to 7. By measurements, the concentration of chlorine dioxide was about 1%, and was to be used for injection within 30 minutes. Injection C containing 15 mg/ml of chlorine dioxide and saline was prepared by the method in Embodiment 1.

Animals: Kunming mice (half male and half female), 18 g to 22 g, SPF.

Dosage, administration route and method: three groups each having five male mice and five female mice, which were subcutaneously injected with injections A, B and C at a dose of 0.3 ml per animal (high) and a dose of 0.1 ml per animal (low) for seven successive days, and then the animals' behavior was observed.

### Results

For the group of injection A, Day 1:, 9 mice at a dose of 0.3 ml per animal (high) died, and 5 mice at a dose of 0.1 ml per animal (low) died; Day 2, 1 mouse at a dose of 0.3 ml per animal (high) died, and 5 mice at a dose of 0.1 ml per animal (low) died.

For the group of injection B, Day 1: 7 mice at a dose of 0.3 ml per animal (high) died, and 4 mice at a dose of 0.1 ml per animal (low) died; Day 2, 2 mice at a dose of 0.3 ml per animal (high) died, and 2 mice at a dose of 0.1 ml per animal (low) died; Day 3: 1 mouse at a dose of 0.3 ml per animal (high) died, and 3 mice at a dose of 0.1 ml per animal (low) died; Day 5: no mice at a dose of 0.3 ml per animal (high) died, and 1 mouse at a dose of 0.1 ml per animal (low) died.

For the group of injection C, from the first day to the seventh day, no mice died.

Conclusion: the injection C is relatively safer. It is indicated that the chlorine dioxide solution as an injection prepared by mixing sodium chlorite with an acid by a conventional method is not safe mainly due to the presence of impurities other than chlorine dioxide after mixing and the low pH. Therefore, the injection containing chlorine dioxide of the present invention is relatively safer or less toxic.

### Embodiment 3: Efficacy Tests of the Injections Containing Chlorine Dioxide

Chlorine dioxide injection: injection A containing chlorine dioxide having a concentration of 2 mg/ml of chlorine dioxide was prepared at room temperature by a conventional method In which Chlorine dioxide gas was generated by a standard method for preparing chlorine dioxide gas having a concentration of 99.9% or more (reacting chlorite with an acid). By bubbling and dissolving the chlorine dioxide gas into a saline solution at room temperature, a chlorine dioxide solution having a concentration of 2 mg/ml (at about 2000 ppm) was prepared. Injection B containing 8 mg/ml of chlorine dioxide and saline was obtained by diluting a solution having a concentration of 15 mg/ml of chlorine dioxide prepared by the method in Embodiment 1. Injection C containing 15 mg/ml of chlorine dioxide and a saline solution was prepared by the method in Embodiment 1.

Animals: C57 male mice, 16-18 g, SPF, provided by the Tested animal Center of the Chinese Analysis Institute for Drugs and Biological Products.

Experimental method: each of mice was subcutaneously inoculated with 3×106 B16 melanoma cells at its right posterior armpit, and mice were randomly divided into four groups each having 10 mice. From the eighth day after the inoculation of melanoma, for three groups of intratumor injection, the mice were intratumorally injected with respective injections A\B\C b at respective doses of 2\8\15 mg/ml and 0.2 ml per animal every four days, total 3 times. For the group of tumor model as a control, the mice were injected with 0.9% of sodium chloride injection (at a dose of 0.2 ml per animal). The experiment lasted for 20 days. The animals (ten mice) in the experimental groups were sacrificed on the fourth day after the final administration. The sacrificed mice were weighed, and tumors were stripped completely from the mice and weighed. The tumor inhibition rate was calculated.

Statistical method: The weight and the tumor weight data were expressed as X̅±S. The comparison between groups was performed by t-test, where p<0.05: significant difference; and p<0.01: very significant difference.

Drug efficacy determination: tumor inhibition rate={1-(average tumor weight T of the administration group/average tumor weight C of the control group)}×100%

### Results

TABLE 1
Effect of intratumor injection of chlorine dioxide on metastatic
melanoma B16 in mice

| | | | | | | Tumor |
|---|---|---|---|---|---|---|
| | | Number | Administration | | Tumor | inhibition |
| | Dose | of | route/Administration | Weight (X ± s, g) | weight | rate |
| Groups | (mg/ml) | animals | frequency | Before After | (X ± s, g) | (%) |
| Model | — | 10 | Q4D it × 3 | 17.9 ± 0.7 | 3.98 ± 0.41 | — |
| control | | | | 19.7 ± 0.9 | | |
| Injection A | 2.0 | 10 | Q4D it × 3 | 17.7 ± 0.4 | 3.01 ± 1.43 | 24.37* |
| | | | | 19.4 ± 1.8 | | |
| Injection B | 8.0 | 10 | Q4D it × 3 | 17.8 ± 0.5 | 1.01 ± 0.23 | 74.62** |
| | | | | 19.5 ± 1.3 | | |
| Injection C | 15.0 | 10 | Q4D it × 3 | 17.9 ± 0.6 | 0.41 ± 0.34 | 89.69** |
| | | | | 19.6 ± 1.0 | | |

Notes:
Q4D means administrating once every 4 days;
*p < 0.05, and
**p < 0.01.

Conclusion: when the injection containing chlorine dioxide was used for treating malignant tumors, the tumor inhibition rate depends on the dose, and the injection has more obvious inhibition effect on tumors at a dose of more than 8 mg/ml. Therefore, the injection containing chlorine dioxide in the present invention has more desirable pharmacological effect.

### Embodiment 4: Effect of the Injection Containing Chlorine Dioxide on Inhibiting Tumors by Tumor Ablation and Stimulation of Response of Immune System

Chlorine dioxide injection: injection A having a concentration of 2.5 mg/ml of chlorine dioxide was prepared at room temperature by a conventional method in which Chlorine dioxide gas was generated by a standard method for preparing chlorine dioxide gas having a concentration of 99.9% or more (reacting chlorite with an acid). By bubbling and dissolving the chlorine dioxide gas into a saline solution at room temperature, a chlorine dioxide solution having a concentration of 2.5 mg/ml (at about 2500 ppm) was prepared. An injection B is an injection containing 8 mg/ml of chlorine dioxide and a saline solution. An solution having a concentration of 15 mg/ml of chlorine dioxide was prepared by the method in Embodiment 1 and then diluted to a concentration of 8 mg/ml with the saline solution. Injection C containing 15 mg/ml of chlorine dioxide and saline was prepared by the method in Embodiment 1.

Animals: C57 male mice, 16-18 g, SPF, provided by the Tested animal Center of the Chinese Analysis Institute for Drugs and Biological Products.

Experimental method: each of mice was subcutaneously inoculated with 1×106 B16 cells at its right posterior armpit, and intravenously inoculated with 5×105 B16 cells to build a lung metastasis model. Mice were randomly divided into 4 groups each having 9 mice. From the eighth day after the inoculation of melanoma, for three groups of intratumor injection, the mice were intratumorally injected with respective injections A\B \C at a dose of 2.5\8\15 mg/ml and 0.2 ml per animal every four days, total 4 times. For the group of tumor model group as a control, the mice were injected with 0.9% of sodium chloride injection solution (at a dose of 0.2 ml per animal). The experiment lasted for 28 days. On the 21st day, 3 mice in each group were detected for inflammatory cell factors. On the 28th day, all animals (6 mice) were sacrificed, and their lung tissues were removed. The number of B16 metastasis in the lung tissues was counted, and the tumor were completely stripped and weighed. The tumor inhibition rate was calculated.

Statistical method: The number of lung metastasis and the tumor weight were expressed as X̅±S. The comparison between groups was performed by t-test, the value of p less than 0.05 indicated a significant difference, where p<0.05: significant difference; and p<0.01: very significant difference.

Drug efficacy determination: tumor inhibition rate={1-(average tumor weight T of the administration group/average tumor weight C of the control group)}×100%

### Results

TABLE 2
Effect of intratumor injection of chlorine dioxide injection on
subcutaneous metastatic melanoma B16 and lung metastases in mice

| | | | | | | | Tumor |
|---|---|---|---|---|---|---|---|
| | | Number | Administration | Number of lung | | Tumor | inhibition |
| | Dose | of | route/Administration | metastasis (X ± s, g)/ | | weight | rate |
| Groups | (mg/ml) | animals | frequency | Significance | | (X ± s, g) | (%) |
| Model | — | 6 | Q4D it × 4 | 15 ± 7 | — | 3.53 ± 0.45 | — |
| control | | | | | | | |
| Injection A | 2.5 | 6 | Q4D it × 4 | 14 ± 7 | — | 2.21 ± 0.53 | 37.39* |
| Injection B | 8.0 | 6 | Q4D it × 4 | 3 ± 1 | ** | 0.71 ± 0.13 | 79.89** |
| Injection C | 15.0 | 6 | Q4D it × 4 | 0 ± 0 | ** | 0.11 ± 0.07 | 96.88** |

Notes:
Q4D means administrating once every 4 days;
*p < 0.05, and
**p < 0.01.

The inflammatory cell factor IL-1β (pg/ml) in peripheral blood was detected by ELISA method, and the tumor necrosis factor NF-α (pg/ml/40 μg protein) in tumor tissues was detected by ELISA method.

TABLE 3
Results of detection of inflammatory cell factor IL-1βand tumor
necrosis factor NF-α in mice in different groups

| Groups | N | IL-1β (X ± s) | TNF-α (X ± s) |
|---|---|---|---|
| Injection C | 3 | 98.75 ± 10.22** | 170 ± 30** |
| Injection B | 3 | 79.34 ± 5.66* | 145 ± 32** |
| Injection A | 3 | 64.60 ± 28.36 | 94 ± 32* |
| Model control | 3 | 60.00 ± 9.644 | 35 ± 12 |

Notes:
*p < 0.05, and **p < 0.01.

The results show, compared to the conventional chlorine dioxide solution having a concentration of 2.5 mg/ml, that the injection containing a high concentration of chlorine dioxide in the present invention can significantly increase the number of cell factors released by antitumor immune cells, thus significantly inhibiting the growth of non-injected tissue tumors the growth of distal tumors and metastatic tumors. It is considered that a high concentration of chlorine dioxide can facilitate the apoptosis or necrosis of tumor cells, which deliver specific antigens to stimulate the recognition of immune cells and generate a corresponding immune response, so that the distal tumors and metastatic tumors are inhibited (Krysko DV, Garg AD, Kaczmarek A, Krysko O, Agostinis P, Vandenabeele P. Immunogenic cell death and DAMPs in cancer therapy. Nat Rev Cancer (2012) 12(12):860-75).

Conclusion: when the injection containing chlorine dioxide was used for treating malignant tumors, the tumor inhibition rate depends on the dose, and the injection has more obvious inhibition effect on tumors at a dose of more than 8 mg/ml. The injection at a dose of 2.5 mg/ml of chlorine dioxide has no inhibition effect on metastatic tumors, while the injection at a dose of more than 8 mg/ml significantly inhibit both tumors at non-injection sites and metastatic tumors. This inhibition is exerted by causing an immune response of the immune system.

### Embodiment 5: Preparation and Preservation Tests of Injection Containing Chlorine Dioxide

28% of sodium chlorite solution and 30% of hydrochloric acid solution were mixed in a reactor equipped with only one gas outlet. The gas outlet of the reactor was connected to PBS (phosphate buffer solution for injection) via a duct. The duct was immersed into the PBS at a temperature of 1° C. which was maintained constant with an external refrigeration device. The reactor containing the mixed sodium chlorite solution and hydrochloric acid solution was heated to 85° C. into which nitrogen was fed (the nitrogen was fed below the liquid surface of the mixed solution through a gas inlet), and the generated chlorine dioxide gas was bubbled into the cold PBS with nitrogen. After a period of time, the chlorine dioxide and the PBS were transferred to a closed glass container, and observed at 20° C. After the chlorine dioxide and the PBS were mixed uniformly, the closed glass container was stored in a refrigerator at 2° C. to 8° C. By measurements, the chlorine dioxide solution having a concentration of 20 mg/ml was prepared. The solution was packaged in 10 ml glass bottles as injections, and then stored away from light at 2° C. to 8° C.

The prepared chlorine dioxide injections in 10 ml glass bottles were stored in dark rooms at 5° C. and 23° C. Comparison of stability of chlorine dioxide injection at different temperatures was made. The content of ClO₂ in the samples after 1 day, 22 days and 100 days was measured by spectrophotometry within 100 days, respectively. Results were shown in Table 4.

TABLE 4
Measurements of ClO₂ in samples

| | | Stored at 5° C. | | Stored at 23° C. | |
|---|---|---|---|---|---|
| 1 | day | 20 | mg/ml (100%) | 20 | mg/ml (100%) |
| 22 | days | 19.6 | mg/ml (98%) | 19 | mg/ml (95%) |
| 100 | days | 18.5 | mg/ml (92.5%) | 13.2 | mg/ml (66%) |

It is indicated that the injection containing chlorine dioxide of the present invention is preferably stored at a low temperature so that the concentration of chlorine dioxide may be reduced less or the injection may be stored for a longer period of time.

### Embodiment 6: Preparation and Preservation Tests of the Injection Containing Chlorine Dioxide

35% of sodium chlorite solution and 40% of sulfuric acid solution were mixed in a reactor equipped with only one gas outlet. The gas outlet of the reactor was connected to another container at −10° C. via a duct, and the duct was inserted into the container in a top-down approach with only air at a temperature of −10° C. which was maintained constant with an external refrigeration device. Nitrogen was fed into the reactor containing the mixed sodium chlorite solution and hydrochloric acid solution (the nitrogen was fed below the liquid surface of the mixed solution through a gas inlet), and the generated chlorine dioxide gas was bubbled into the low-temperature container with nitrogen. After a period of time, the chlorine dioxide was attached to the bottom of the container in solid or liquid form. The chlorine dioxide was transferred to a closed glass or metal container by a standard method, and then the closed glass or metal container was stored in a refrigerator at −20° C. to −10° C. By measurements, the prepared chlorine dioxide was chlorine dioxide having a concentration approximate to 100%. The chlorine dioxide was packaged in 10 ml glass bottles as injections, with 0.2g pure chlorine dioxide per bottle. The glass bottles were stored away from light at −20° C. to −10° C. When the chlorine dioxide is administrated to a patient as an injection, the chlorine dioxide is diluted with a saline solution and injected immediately. For example, 10 ml of saline solution is injected into the 10 ml glass bottle with chlorine dioxide to obtain a chlorine dioxide injection. After the chlorine dioxide is uniformly dissolved in the saline solution, the injection can be injected into the body immediately.

The prepared chlorine dioxide injection packaged in 10 ml glass bottles was stored in a dark room at −10° C. and 5° C. Comparison of stability of chlorine dioxide injection at different temperatures was made Weight of ClO₂ in samples after 1 day, 22 days and 100 days was measured with a precision balance, respectively, and the results were shown in Table 5.

TABLE 5
Weight of ClO₂ in samples

| | | Stored at −10° C. | Stored at 5° C. |
|---|---|---|---|
| 1 | day | 200 mg (100%) | 200 mg (100%) |
| 22 | days | 198 mg (99%) | 190 mg (95%) |
| 100 | days | 192 mg (96%) | 168 mg (84%) |

It is indicated that the injection containing a high concentration of chlorine dioxide in the present invention is preferably stored at a low temperature. Particularly, the chlorine dioxide injection of non-solution form is more stable when stored at −10° C. than stored at 5° C.

### Embodiment 7: Preparation of Injections Containing 3 mg/ml, 5 mg/ml and 25 mg/ml of Chlorine Dioxide

28% of sodium chlorite solution and 50% of citric acid solution were mixed in a reactor, and then introduced to a saline solution containing 0.9% of sodium chloride at 1° C. via a duct, with the inlet of the duct immersed in the saline solution. The reactor was heated for 30 minutes at a constant temperature of 90° C., and chlorine dioxide was bubbled into the saline solution which was always maintained at a temperature of 1° C. with an external device. After 40 minutes, the mixed solution was transferred to a closed container and then placed at 20° C. After the chlorine dioxide and the saline solution were mixed uniformly, the mixed solution was placed in a refrigerator at 2° C. to 8° C. for storage. By measurements, a chlorine dioxide solution having a concentration of 25 mg/ml was prepared. This chlorine dioxide solution was diluted with a saline solution to obtain a chlorine dioxide solution having a concentration of 3 mg/ml and a chlorine dioxide solution having a concentration of 5 mg/ml. The solution can be packaged in 10 ml glass bottles as injections, and then stored away from light at 2° C. to 8° C.

What is claimed is:

1. An injection containing chlorine dioxide, wherein the injection is composed of chlorine dioxide and an additive suitable for injections; and a concentration of chlorine dioxide in the injection when used is 3-25 mg/ml.

2. The injection of claim 1, wherein the injection is stored away from light at −20° C. to 11° C.

3. The injection of claim 2, wherein the injection is stored away from light at −18° C. to 9° C.

4. The injection of claim 3, wherein the injection is stored away from light at 2° C. to 8° C.

5. The injection of claim 1, wherein the concentration of chlorine dioxide in the injection when used is 5-25 mg/ml.

6. The injection of claim 5, wherein the concentration of chlorine dioxide in the injection when used is 8-20 mg/ml.

7. The injection of claim 1, wherein the injection of a concentrated solution is diluted for use.

8. The injection of claim 1, wherein administration of the injection to a target organ or tissue is intravenous, arterial, intramuscular, subcutaneous, intradermal, intracardiac, intraperitoneal, intrathecal, intra-articular, puncture, rectal, sublingual, nasal, transdermal, by inhalation or topical.

9. The injection of claim 1, wherein the injection comprises forms of a suspension and an emulsion.

10. The injection of claim 1, wherein the injection is extended to forms selected from the group consisting of ointment, inhalant, liquor, suppository, patch, cataplasm, lotion for external use, tablet, liquid preparation, capsule, granule, powder, pill, syrup and lozenge.

11. A method for producing the injection of claim 1, comprising:
1) generating chlorine dioxide gas;
2) introducing the chlorine dioxide gas into a medium at −20° C. to 11° C. via a duct; and
3) when the medium is a gaseous medium, collecting chlorine dioxide to obtain a chlorine dioxide injection having a concentration of chlorine dioxide of 99.9% or more; and when the medium is a liquid medium, raising temperature of the liquid medium to less than 20° C. to obtain the injection containing chlorine dioxide after the chlorine dioxide is uniformly dissolved in the liquid medium.

12. The method of claim 11, wherein in step 3, the liquid medium for absorbing the chlorine dioxide gas is a saline solution at 1° C. to 5° C.

13. The method of claim 11, wherein in step 2, the chlorine dioxide gas is introduced into the medium via the duct in a top-down approach.

14. The method of claim 11, wherein the medium is a liquid medium, and a additive is added in the liquid medium before or after the introduction of the chlorine dioxide gas.
